(19) United States
(12) Patent Application Publication
(10) Pub. No.: US 2008/0075799 A1
(43) Pub. Date: Mar. 27, 2008

(54) Use Of Medicinal Plants And Extracts Thereof With Growth Promoting Activity In Veterinary Medicine And Zootechnics

(75) Inventors: Andrea Giori, Milano (IT); Gianpaolo Forni, Milano (IT); Giancomo Mombelli, Milano (IT)

Correspondence Address: YOUNG & THOMPSON, 745 SOUTH 23RD STREET, 2ND FLOOR, ARLINGTON, VA, 22202, US

(73) Assignee: INDENA S.P.A., Milano (IT)

(21) Appl. No.: 11/659,333

(22) PCT Filed: Jul. 20, 2005
(86) PCT No.: PCT/EP05/07896

§ 371 Date: Apr. 9, 2007

(30) Foreign Application Priority Data
Aug. 6, 2004 (IT) MI2004A 001631

(51) Int. Cl. A61K 36/815; A61P 3/00

(52) U.S. Cl. 424/777

(57) ABSTRACT

The present invention relates to the use of extracts of plants or fruits of the Lycium genus, in particular of Lycium barbarum, for the preparation of growth promoters for use in livestock.

The present invention relates to the use of extracts of plants or fruits of the genus Lycium, in particular of Lycium barbarum, for the preparation of growth promoters for use in livestock.

### TECHNOLOGICAL BACKGROUND

Bacterial antibiotic resistance is an increasing problem which affects public health, including the veterinary field.

The use in veterinary medicine of antibiotics that are mostly the same as or strictly related to those used in human medicine, and the established practice of use of antibiotics at sub-therapeutical doses as growth promoters and mass metaphylactic agents, are the main causes of diffusion of acquired antibiotic resistance by a number of bacterial species responsible for pathologies in domestic animals and often even in humans. The presence of antibiotic residues in meat and, indirectly, the selection of antibiotic-resistant bacteria, which can transfer the resistance genes to bacteria pathogenic to humans, can be a hazard to human health.

Approximately 25% of the total antibiotic production is intended for use in livestock, the 90% of them being used at sub-therapeutical doses for both the prevention of diseases and the promotion of growth. Antimicrobials have been added to feed and extensively used in zootechnics since the '50s to improve productive performances. In swine breeding, for instance, weaning of piglets is the most delicate step, as the distressing events related to the transfer and mixing of different groups of animals and the changes of feed induce alterations in the intestinal microbial flora of the animal, making it more susceptible to infections. The use of antibiotics in the prophylaxis of endemic diseases occurring during weaning has lead to a generalized use of antibiotics which are included in feed for times which can be protracted until the animal is even 70-80 day old.

As a consequence of antibiotic-resistance emergency, the European Union has followed a restricted approach concerning the use of antibiotics as growth promoters, antimicrobial prophylaxis, multi-therapies, mass medication through feed and water. In particular, EU is withdrawing all authorizations concerning the use of antibiotics as growth promoters by 2006. However, the prohibition of use of antibiotics as growth promoters and prophylactic agents will involve a deterioration of animals health, with an increase in diarrhoea, weight loss and mortality.

Different approaches exist for reducing the dependency of zootechnics, more particularly swine breeding, on the use of antibiotics: the choice one is the development of alternatives acting through the control of bacteria and promoting the natural reactivity of the animal, thereby inducing an improvement in health as well as the optimization of performances. Mandatory characteristics for such alternative solutions are safety of use and cheapness for producers, and hence for consumers.

Lycium is a shrub native to China, belonging to the Solanaceae class, which produces small oval fruits which turn orange/red in the autumn.

Fruits of plants of the Lycium genus have been used for a long time, in the form of aqueous extracts, in Chinese traditional medicine for the treatment of a variety of conditions including kidney and liver disorders, anaemia, dizziness and tinnitus.

### DISCLOSURE OF THE INVENTION

It has now been found that the administration of parts of plants, particularly fruits or extracts of fruits of plants of the Lycium genus, in particular Lycium barbarum, to livestock, is at least as much as active as the administration of growth-promoting antibiotics.

Therefore, the present invention relates to compositions for the veterinary or zootechnical use, comprising as active ingredients ground fruits or extracts of fruits of Lycium, preferably of Lycium barbarum, admixed with suitable carriers or in the form of feed supplements.

Said compositions proved capable of promoting growth in livestock, in particular swine, bovine, ovine and equine, more particularly swine, without inducing the drawbacks and side effects of growth-promoting antibiotics.

Dosages will depend on a number of factors, such as species, size, weight and age of the animal. In principle, a typical dosage will range from 0.1 g to 10 g of extract/fruits per animal/day.

The extracts usable according to the invention can be prepared by a process which comprises repeated extractions of Lycium barbarum fruits with water. The extraction temperature can range from 5° C. to water boiling temperature, preferably 90° C. The resulting extracts are combined and concentrated to small volume under vacuum, then dried in a static dryer under vacuum at 60° C.

The following examples illustrate the invention in further detail.

### EXAMPLE 1

### Preparation of a Lycium barbarum Fruits Extract for the Zootechnical Use

1 kg of dry Lycium barbarum fruits is placed in a jacketed percolator and covered with 1.9 L of water for 3 hours at a temperature of 90° C. The first extract is recovered and 4 more extractions are carried out, using 7.1 L of water totally. The filtered and combined extracts (6.2 L) are concentrated to small volume under vacuum then dried in a static dryer at 60° C. under vacuum. The resulting product is ground, thereby obtaining 650 g of dry whole extract.

### EXAMPLE 2

### In Vivo Test

Experiments were carried out on 120 piglets divided into 4 groups of 30 animals each.

Group 1 (control) was treated with standard feed free from additives. Group 2 (antibiotic) was treated with medicated feed containing 2 g of apramycin and 1 g of colistin per kg of feed. Group 3 was treated with standard feed added with 2 g/Kg of Lycium extract. Group 4 was treated with standard feed added with 5 g/kg of finely ground Lycium fruits.

All animals were treated during weaning from day 22 to day 41.

The animal well being was evaluated by the total weight increase, the feed daily intake and the percentage of diarrhoeic animals. Moreover, from day 41, faeces samples were microbiologically analysed for bacterial counts of total mesophyte charge, total Coliform and Lactobacilli. All parameters were evaluated with standard methods known to those skilled in the art.

Results reported in the following Tables prove that the addition to standard feed of Lycium in the form of both finely ground roots and water-ethanol extract, although being devoid of per se in vitro antibacterial activity, can be a valuable replacement for antibiotic growth promoters in weaning foodstock.

TABLE 1
Piglets weights and Mean Weight Increase (MWI) on day 41.

Piglets weight (kg)

| Treatment | day 22 | day 41 | MWI |
|---|---|---|---|
| Control | 7.02 ± 1.2 | 10.25 ± 1.5 | 3.22 |
| Antibiotic | 7.43 ± 1.3 | 12.21 ± 1.6 | 4.78 |
| Lycium fruits | 7.27 ± 1.2 | 11.23 ± 1.4 | 3.96 |
| Lycium aqueous extract | 7.22 ± 1.3 | 11.56 ± 1.3 | 4.34 |

TABLE 2
Mean daily intake: amount of feed daily intake, expressed
in kg, between days 22 and 41 of treatment.

| Treatment | kg |
|---|---|
| Control | 0.22 |
| Antibiotic | 0.37 |
| Lycium fruits | 0.33 |
| Lycium aqueous extract | 0.35 |

TABLE 3
Effect of treatment on diarrhoea onset
in the various treated groups.

| Treatment | % of animals with diarrhoea |
|---|---|
| Control | 60 |
| Antibiotic | 10 |
| Lycium fruits | 30 |
| Lycium aqueous extract | 10 |

TABLE 4
Effect of administration of the tested substances on different bacterial
populations (log Ufc/g) from faeces samples taken on day 41.

| Treatment | E. Coli | CBT | Lactobacilli |
|---|---|---|---|
| Antibiotic | 6.41 | 6.47 | 5.38 |
| Control | 8.05 | 7.55 | 6.93 |
| Lycium fruits | 6.79 | 7.02 | 6.93 |
| Lycium aqueous extract | 6.56 | 6.70 | 5.99 |

1. A method of promoting growth in an animal comprising administering an effective amount of parts or extracts of plants of the Lycium genus to an animal in need thereof.

2. The method according to claim 1 wherein the plant of the Lycium genus is Lycium barbarum.

3. The method according to claim 1, wherein the parts of Lycium are the fruits.

4. The method according to claim 1, wherein the extract is a fruits aqueous extract.

5. Veterinary or zootechnical compositions comprising parts or extracts of plants of the Lycium genus in admixture with suitable carriers.

6. Compositions as claimed in claim 5 comprising fruits or aqueous extracts of Lycium barbarum fruits.

7. Feed comprising fruits or fruits extracts of plants of the Lycium genus, particularly Lycium barbarum.

8. The method according to claim 2, wherein the parts of Lycium are the fruits.

9. The method according to claim 2, wherein the extract is a fruits aqueous extract.

10. The method according to claim 1, wherein the parts or extracts of the plants of the lycium genus are in admixture with suitable carriers

11. The method according to claim 1, wherein the effective amount is 0.1 g TO 10 g of extract/root per animal/day.
